(19) United States
(12) Patent Application Publication
(10) Pub. No.: US 2026/0083466 A1
(43) Pub. Date: Mar. 26, 2026

(54) Catheter System for Treating Calcified Plaque

(71) Applicant: Medtronic Vascular, Inc., Santa Rosa, CA (US)

(72) Inventors: Aram Jamous, Athenry (IR); Meriam Zahraa W. Jassim, Limerick (IR); Cian Walsh, Galway (IR); Eoghan D. Twohig, Wicklow Town (IR); Bryan S. Hansen, Oranmore (IR); Michael P. Rodgers, Westport (IE)

(21) Appl. No.: 19/111,217

(22) PCT Filed: Sep. 12, 2023
(86) PCT No.: PCT/US2023/032537

§ 371 Date: Mar. 12, 2025

Related U.S. Application Data
(60) Provisional application No. 63/375,313, filed on Sep. 12, 2022.

(51) Int. Cl. A61B 17/22; A61B 17/00

(52) U.S. Cl. CPC A61B 17/22; A61B 2017/00477; A61B 2017/00539; A61B 2017/22054; A61B 2017/22079

(57) ABSTRACT

A catheter system may include a longitudinal pressure wave generator that generates a longitudinal pressure wave. The longitudinal pressure wave generator may be couplable to the proximal end portion of a catheter body to enable delivery of the generated longitudinal pressure wave to the catheter body. The catheter body may be configured to enable the longitudinal pressure wave from the longitudinal pressure wave generator to propagate therethough toward the distal end portion of the catheter body and deliver the propagated longitudinal pressure wave to the calcified plaque at the treatment site. A catheter system may include a treatment member body and a plurality of fins coupled to the treatment member body. The plurality of fins are deployable using an actuator.

### FIELD

The present technology is generally related to a catheter system and method for treating calcified plaque within a body of a subject.

### BACKGROUND

A variety of techniques and instruments have been developed to percutaneously treat calcified plaque within a body of a subject. As an example, calcified plaque may build up within the circulatory system of the subject. A common example is the buildup of fatty deposits (atheromas) in the intimal layer (under the endothelium of a patient's blood vessels). Over time, what is initially deposited as relatively soft, cholesterol-rich atheromatous material often hardens into a calcified atherosclerotic plaque. The atheromas may be referred to as stenotic lesions or stenoses while the blocking material may be referred to as stenotic material. If left untreated, such stenoses can so sufficiently reduce perfusion that angina, hypertension, myocardial infarction, strokes or the like may result. Angioplasty or atherectomy may be performed to improve blood flow. However, the presence of calcified plaque typically leads to difficulty in adequately treating the blood vessel.

### SUMMARY

The techniques of this disclosure generally relate to a catheter system and method for treating calcified plaque within a body of a subject.

In one aspect, the present disclosure provides a catheter system for treating calcified plaque within a body of a subject. The catheter system comprises a catheter body having opposite proximal and distal end portion and a longitudinal axis extending therebetween, wherein the catheter body is configured to be percutaneously inserted into the body of the subject and delivered to a treatment site having calcified plaque.

As configured to generate a longitudinal pressure wave, wherein the longitudinal pressure wave generator is couplable to the proximal end portion of the catheter body enable delivery of the generated longitudinal pressure wave to the catheter body. The catheter body is configured to enable the longitudinal pressure wave from the longitudinal pressure wave generator to propagate therethough toward the distal end portion of the catheter body and deliver the propagated longitudinal pressure wave to the calcified plaque at the treatment site.

In another aspect, the disclosure provides a method of treating calcified plaque at a treatment site within a body of a subject. The method comprises delivering a catheter body of a catheter system to the treatment site so that a distal end portion of the catheter body is adjacent the calcified plaque; propagating a longitudinal pressure wave through the catheter body from a proximal end portion toward a distal end portion of the catheter body; and delivering the longitudinal pressure wave from the distal end portion of the catheter body to the calcified plaque.

In yet another aspect, the disclosure provides a catheter system for treating calcified plaque within a body of a subject. The catheter system comprises a catheter body having opposite proximal and distal end portion and a longitudinal axis extending therebetween. The catheter body is configured to be percutaneously inserted into the body of the subject and delivered to a treatment site having calcified plaque. A treatment member is at the distal end portion of the catheter body. The treatment member includes a treatment member body and a plurality of fins coupled to the treatment member body. The treatment member is selectively configurable between a non-deployed configuration, in which the fins are generally flush with the treatment member body, and a deployed configuration, in which the fins project radially outward from the treatment member body. An actuator is configured to enable a user to configure the treatment member between the non-deployed and deployed configurations.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a schematic of an example of a catheter system for treating calcified plaque showing propagating longitudinal pressure waves.

FIG. 2 is schematic of a distal end portion of the catheter system treating calcified plaque within a body lumen.

FIG. 3 is a schematic of a longitudinal pressure wave.

FIG. 4 is a schematic of another example of a catheter system showing a distal end portion of the catheter system treating calcified plaque in a body lumen.

FIG. 5 is a schematic of yet another example of a catheter system showing a distal end portion of the catheter system treating calcified plaque in a body lumen.

FIG. 6 is a schematic of another example of a catheter system including a treatment member at a distal end thereof, the treatment member being in a non-deployed configuration.

FIG. 7 is a cross section of the distal end portion of the catheter system of FIG. 6.

FIG. 8 is similar to FIG. 6 but with the treatment member in a deployed configuration.

FIG. 9 is a cross section of the distal end portion of the catheter system of FIG. 8.

### DETAILED DESCRIPTION

The following description generally relates to examples of a treatment catheter system for treating calcified plaque within a body of a subject. The illustrated examples are suitable for treating calcified plaque within a circulatory system of the subject, such as blood vessels and/or the heart of the subject. The illustrated examples may also be suitable for treating other body lumens outside the circulatory system.

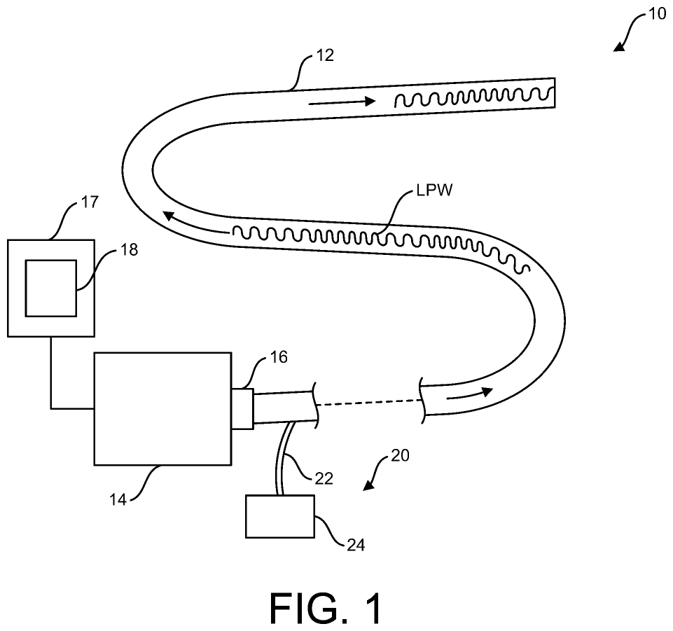

Referring to FIG. 1, one example of a catheter system for treating calcified plaque within a body of a subject is generally indicated at reference numeral 10. In general, the catheter system 10 is configured to deliver a longitudinal pressure wave to the calcified plaque. The longitudinal pressure wave modifies or disrupts the calcified plaque. For example, the longitudinal waves may fracture the calcified plaque, thereby facilitating treatment. In one example, the calcified plaque may be treated further, such as through angioplasty or atherectomy or other treatments, or the treatment using longitudinal waves may be the primary or only treatment of the calcified plaque.

Referring still to FIG. 1, the catheter system 10 includes a catheter body 12 having proximal and distal end portions and a longitudinal axis extending therebetween. The catheter body 12 is designed and constructed to be percutaneously inserted into a blood vessel of the subject to deliver the distal end portion of the catheter body to the treatment site including calcified plaque. As non-limiting examples, the catheter body 12 may have a length from about 150 mm to about 400 mm, and a diameter from about 0.014 in (0.3556 mm) to about 0.120 in (3.048 mm). The catheter body 12 may suitably comprise a flexible material, such as plastic, to enable the body to traverse a tortuous path to the treatment site.

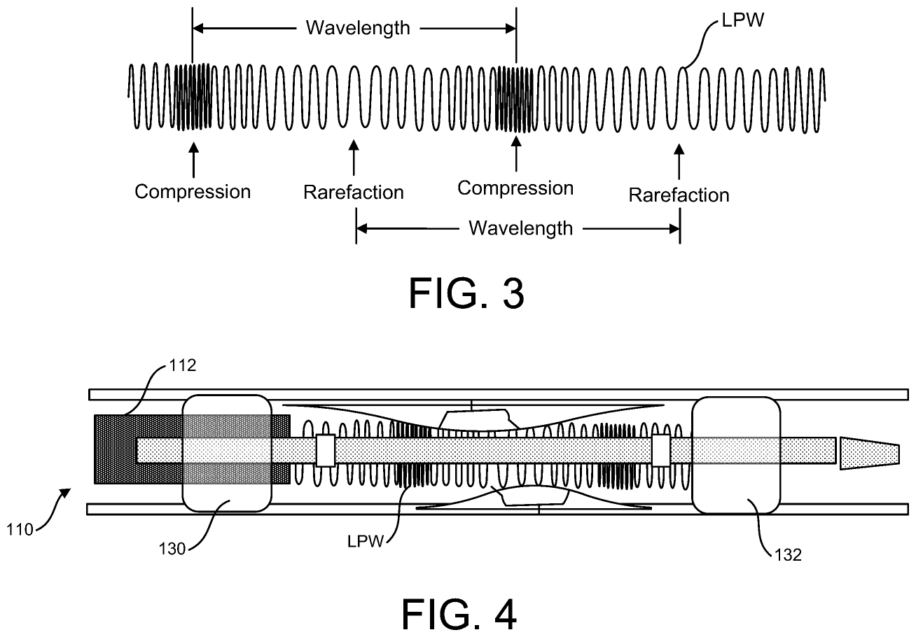

A longitudinal pressure wave generator 14 of the catheter system 10 is configured to generate at least one longitudinal pressure wave LPW. The generator 14 is operatively coupled or couplable to the proximal end portion of the catheter body 12 to deliver the generated longitudinal pressure wave LPW to the catheter body. The generator 14 is external to the patient and may be any suitable device for producing the longitudinal pressure wave LPW. For example, the generator 14 may include an apparatus for producing hydraulic shock (i.e., water hammer). In such a system the longitudinal pressure wave LPW is generated by flowing fluid (e.g., liquid) abruptly stops or changes direction. Other ways of generating the longitudinal pressure wave LPW includes i) use of a piston(s); ii) use of a mechanical trigger or hammer action; and/or iii) a controlled explosion or rapid expansion of compressed gas (e.g., air). A suitable longitudinal pressure wave generator 14 is described in U.S. Ser. No. 14/655,639, filed Jun. 25, 2015, the entirety of which is hereby incorporated by reference herein. A typically pressure wave generated by the pressure wave generator 14 is shown in FIG. 3.

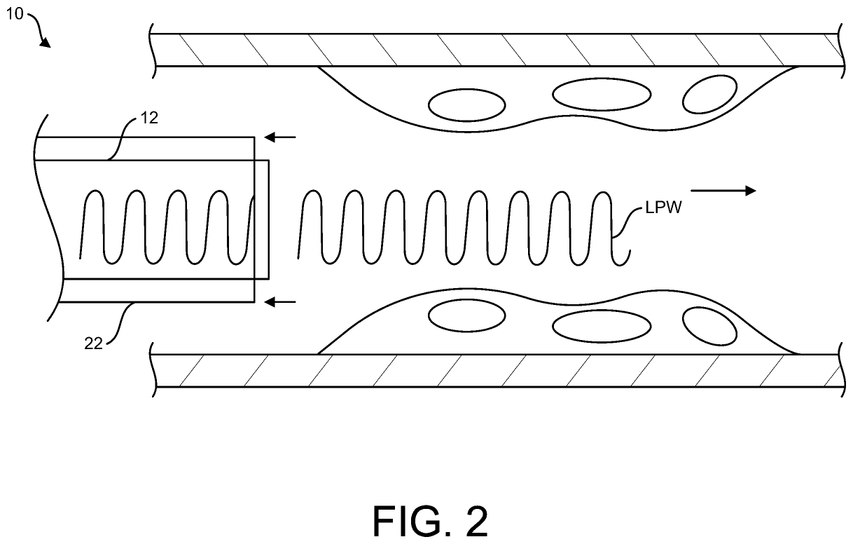

Referring to FIGS. 1 and 2, the generated longitudinal pressure wave LPW propagates from the generator 14 and into the proximal end portion of the catheter body 12. A coupling or interface 16 may couple the catheter body 12 to the generator 14 to enable transmission of the longitudinal pressure wave LPW into the catheter body 12. The generated pressure wave propagates longitudinally through the catheter body 12 toward the distal end portion via a medium (e.g., blood, saline, or other suitable medium) disposed in an internal passage of the catheter body 12. Sidewall(s) of the catheter body 12 may be configured to limit energy loss as the pressure wave propagates toward the distal end of the catheter body 12. For example, the wall(s) of the catheter body 12 may include a suitable insulating and/or reflective material to reduce or limit absorption by or transmitting of pressure wave LPW through the sidewall(s) of the catheter body 12 to limit energy loss within the catheter body 12 as the pressure waves propagate distally.

As shown in FIG. 2, the propagating pressure wave exits the distal end portion of the catheter body 12 to direct the wave energy toward the calcified plaque. In one example, the distal end of the catheter body 12 is configured to enable delivery of the pressure wave energy to the calcified plaque. In one example, the distal end is open so that the wave energy propagates directly into the fluid (e.g., blood) of the treatment site and/or the calcified plaque. In another example, the distal end may include an interface material different from the material of the sidewall(s) of the catheter body 12 to facilitate transmission of the energy through the distal end of the catheter body 12. In one example, the interface material may have an impedance substantially matching or similar to the impedance of the medium inside the catheter body 12 to limit refraction of the pressure wave. The impedance may also substantially match or be similar to the impedance of the fluid at the treatment site to limit refraction. Propagation of the wave energy through the fluid at the treatment site is suitable to modify or disrupt the calcified plaque.

The user may control activation of the pressure wave using a control unit 17. The control unit 17 may include a controller (e.g., processor and memory) and a user interface 18. The controller communicates with the longitudinal pressure wave generator 14 and the user interface 18 enables communication between the user and the controller. The user interface 18 may include a touch screen or other interface. The energy of the pressure wave generated by the generator 14 may be tuned or calibrated to produce a desired, safe effect on the calcified plaque at the treatment site. As an example, the desired energy may be tuned by adjusting one or more parameters of the pressure wave, including but not limited to frequency and wavelength to produce the desired velocity of the pressure wave. The energy of the pressure wave can be selectively adjustable by the user during the medical procedure, such as through the user interface 18, or the energy of the pressure wave may be fixed and non-adjustable. The generator 14 may be calibrated before the procedure to produce the desired energy.

In one example, the catheter system 10 may further include a material removal device, such as an aspirator 20, for removing any debris (e.g., tissue) detached from the treatment site during treatment. The aspirator 20 may include an aspiration lumen 22 extending along the catheter body 12 from adjacent the distal end portion to the proximal end portion. A source of negative pressure 24 (e.g., a vacuum source) is in fluid communication with the proximal end portion of the aspiration lumen 22 and configured to draw the detached debris into the aspiration lumen and to a collection container.

Referring to FIG. 4, another embodiment of a catheter system for treating calcified plaque within a body of a subject is generally indicated at reference numeral 110. Similar to the first catheter system 10, the present catheter system 110 is configured to deliver longitudinal pressure wave LPW to the calcified plaque. The longitudinal pressure wave modifies or disrupts the calcified plaque. A catheter body 112, a pressure wave generator (not shown), a control unit (not shown), and an aspirator (not shown) may be similar or identical to the respective catheter body 12, pressure wave generator 14, and control 16.

The catheter system 110 further includes at least one barrier (e.g., first and second barriers 130, 132) at the distal end portion of the catheter body 112 configured to engage the body lumen (e.g., blood vessel) adjacent to the treatment site to direct the wave energy toward the calcified plaque. In the illustrated example, the first and second barriers 130, 132 are spaced apart proximal and distal barriers, respectively. As an example, the barriers 130, 132 may be balloons that are selectively inflatable or expandable to engage the inner surface of the body lumen. The proximal balloon 130 may be adjacent to and proximal of the distal end of the catheter body 112 (or generally proximal of the location where the pressure wave exits the catheter system at the treatment site). The distal balloon 132 is distal of and spaced apart from the distal end of the catheter body 112 (or generally distal of the location where the pressure wave exits the catheter system at the treatment site). The distal balloon 132 may be disposed on an extension shaft 136 extending from the distal end portion of the catheter body 112. The inflated proximal and distal balloons 130, 132 within the body lumen enable the wave energy to enter a suitable medium (e.g., a suitable fluid) for propagating toward the calcified plaque. The balloons 130, 132 essentially produce a propagation chamber for the pressure wave.

In one example, the catheter system 110 may be configured to allow a suitable medium to be delivered to the formed propagation chamber. The medium may be the same medium that is present in the catheter body 112. In one example, a suitable flowable medium (e.g., liquid medium) may be injected into the catheter body 112 delivered through the distal end of the catheter system into the formed propagation chamber between the balloons 130, 132. In one example, when inflated or expanded, the balloons 130, 132 may be configured to limit or substantially inhibit the pressure wave energy from transmitting through the balloons. In this way, as shown in FIG. 4, the energy is substantially contained within the formed propagation chamber and the treatment site.

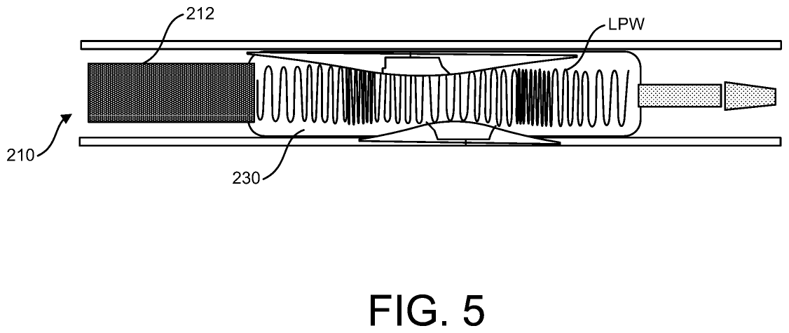

Referring to FIG. 5, another embodiment of a catheter system for treating calcified plaque within a body of a subject is generally indicated at reference numeral 210. Similar to the first catheter system 10, the present catheter system 210 is configured to deliver longitudinal pressure wave LPW to the calcified plaque. The longitudinal pressure wave LPW modifies or disrupts the calcified plaque. A catheter body 212, a pressure wave generator (not shown), a control unit (not shown), and an aspirator (not shown) may be similar or identical to the respective catheter body 12, longitudinal pressure wave generator 14, and control 16.

The catheter system 210 further includes an inflatable or expandable balloon 230 (e.g., angioplasty balloon) configured to direct the wave energy toward the treatment site. The balloon 230 defines an interior configured to be filled with a fluid medium. The distal end portion of the catheter body 212 is in communication with the fluid medium in the balloon 230 so that the wave energy from the catheter body 212 propagates through the balloon and to the treatment site. Distal and/or proximal ends of the balloon 230 may be configured to limit or substantially inhibit the pressure wave energy from transmitting therethrough so that the wave energy is contained within the balloon. The balloon 230 may be configured to apply a suitable pressure on calcified plaque for angioplasty treatment simultaneously with wave energy treatment. Alternatively, the balloon 230 may be configured to apply nominal pressure not suitable for angioplasty but enough to enable transmission of the wave energy to the calcified plaque.

In an exemplary treatment method using any one of the catheter systems 10, 110, 210, the catheter body 12, 112, 212 is inserted into the subject's body and traversed to the treatment site. Using any one of the catheter systems 110, 210, the balloon(s) 130, 132, 230 is then inflated. The wave generator 14 is then activated to deliver longitudinal wave energy to the distal end portion of the catheter body 12, 112, 212 and to the calcified plaque. The wave energy modifies or disrupts the calcified plaque. For example, the energy may fracture (e.g., cause micro-fractures) the calcified plaque. After treatment with the catheter system 10, 110, 210, in one example the catheter body 12, 112, 212 may be withdrawn and subsequent treatment (e.g., angioplasty and/or atherectomy and/or drug treatment) may be performed.

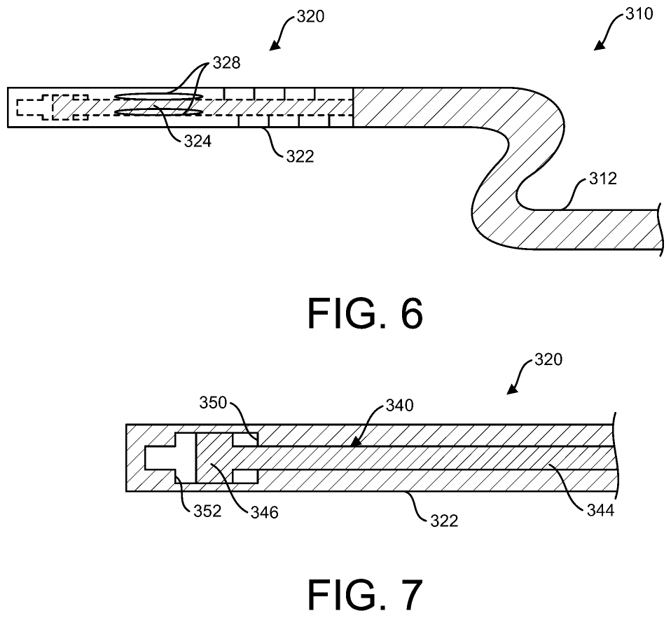

Referring to FIGS. 6-9, another embodiment of a catheter system for treating calcified plaque within a body of a subject is generally indicated at reference numeral 310. The catheter system 310 includes a catheter body 312 having proximal and distal end portions and a longitudinal axis extending therebetween. The catheter body 312 is designed and constructed to be percutaneously inserted into a blood vessel of the subject to deliver the distal end portion of the catheter body to the treatment site including calcified plaque. As non-limiting examples, the catheter body 12 may have a length from about 150 mm to about 400 mm, and a diameter from about 0.014 in (0.3556 mm) to about 0.120 in (3.048 mm). The catheter body 312 may suitably flexible (e.g., a coiled shaft) to enable the body to traverse a tortuous path to the treatment site. As explained below, in one example the catheter 310 is suitable for use as a guidewire for delivering another treatment catheter to the treatment site. In other examples, the catheter 310 may not be used as a guidewire.

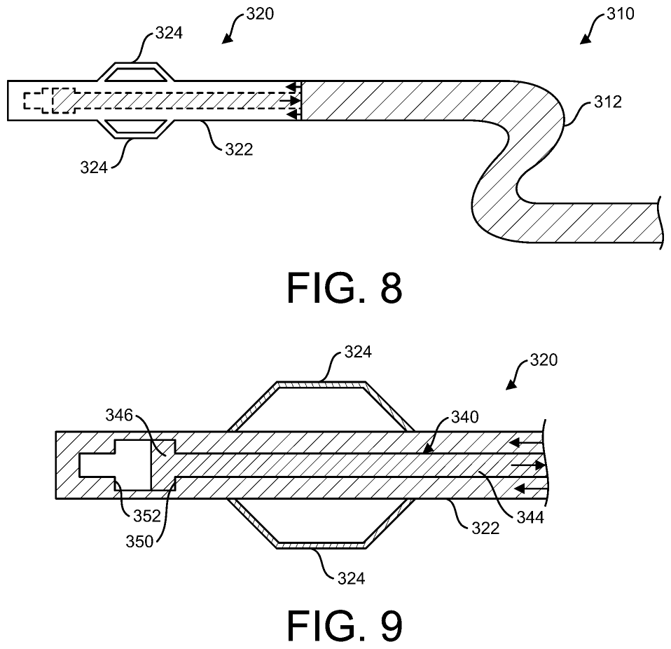

The catheter 310 includes a selectively deployable treatment member 320 at the distal end portion of the catheter body and configured to modify or disrupt the calcified plaque. The treatment member is shown in a deployed configuration in FIGS. 8 and 9 and in a non-deployed configuration in FIGS. 6 and 7. In one example, the treatment member 320 include a body 322 and a plurality of fins 324 that project radially outward relative to the body 322 in the deployed configuration and are generally flush with or recessed relative to the body in the non-deployed configuration. The fins 324 are circumferentially spaced apart from one another around the body 322. In one example, the body 322 and the fins 324 are integrally formed as a one-piece component, with the fins coupled to the body by living hinges enabling the fins to pivot radially outward from the body. As shown in FIG. 6, the treatment member 320 may be formed of a metal tube (e.g., hypotube) with the fins formed by circumferential slots 328 in the tube. The treatment member may be of other configurations and designs and may be formed in other ways.

Referring to FIGS. 7 and 9, an actuator 340 operable by the user selectively configures the treatment member 320 between the deployed and non-deployed configurations. In the illustrated embodiment, the actuator 340 includes a shaft 344 extending along and within the treatment member 320 and the catheter body 312. The shaft 344 is longitudinally movable within and relative to the treatment member 320. A head 346 at the distal end portion of the shaft 344 is engageable with proximal and distal internal shoulders or stops 350, 352 within the treatment member 320. In the illustrated embodiment, the head 346 is threadably coupled within the treatment member 320 and the shaft 344 and head are axially rotatable relative to treatment member 320 and the body 312. Rotation of the shaft 344 and the head 346 in a first direction imparts longitudinal movement of the head and shaft in the proximal direction. Rotation of the shaft 344 and the head 346 in a second direction imparts longitudinal movement of the head and the shaft in the distal direction.

Movement of the actuator 340 proximally relative to the treatment member 320, such as by rotating the shaft 344, causes the head 346 to engage the proximal shoulder 350, thereby applying a compressive force on the treatment member and configuring the treatment member from the non-deployed configuration to the deployed configuration. In other words, the compressive force causes the treatment member 320 to contract longitudinally and the fins to buckle about the living hinges such that the fins 324 project radially outward in the deployed configuration. Movement of the actuator 340 distally relative to the treatment member 320, such as by rotating the shaft 344, causes the head 346 to engage the distal shoulder 352, thereby applying a tensile force on the treatment member and configuring the treatment member from the deployed configuration to the non-deployed configuration. In other words, the tensile force causes the treatment member to expand longitudinally and the fins to retract radially inward about the living hinges. The rotary-to-linear mechanism (e.g., threaded coupling) inhibits back-off, provides a mechanical advantage in applying the proximal and distal forces to the respective shoulders 350, 352, and enables the user to select the amount of radial displacement of the fins 324 and/or force applied to the calcified plaque by the fins.

In one embodiment, the catheter system 310 is designed and constructed as a guidewire. After delivering the guidewire 310 to the treatment site with the treatment member 320 in the non-deployed configuration, the user operates the actuator 340 to deploy the treatment member. As an example, the user may manually rotate the shaft 344 using a control handle or some other device outside the subject's body. Upon deploying the treatment member 320, the fins 324 are moved radially outward and engage the calcified plaque. The fins 324 apply a radially compressive force to the calcified plaque to modify or disrupt the plaque. After treatment with the treatment member 320, the user operates the actuator 340 to retract the fins 324 radially inward to the non-deployed configuration. The guidewire 310 may remain within the body lumen for receiving another catheter system, such as for angioplasty or atherectomy for example.

The invention may be further described by reference to the following numbered paragraphs:
1. 1. A catheter system for treating calcified plaque within a body of a subject, the catheter system comprising:
   a catheter body having opposite proximal and distal end portion and a longitudinal axis extending therebetween, wherein the catheter body is configured to be percutaneously inserted into the body of the subject and delivered to a treatment site having calcified plaque; and
   a longitudinal pressure wave generator configured to generate a longitudinal pressure wave, wherein the longitudinal pressure wave generator is couplable to the proximal end portion of the catheter body to enable delivery of the generated longitudinal pressure wave to the catheter body,
   wherein the catheter body is configured to enable the longitudinal pressure wave from the longitudinal pressure wave generator to propagate therethough toward the distal end portion of the catheter body and deliver the propagated longitudinal pressure wave to the calcified plaque at the treatment site.
2. The catheter system set forth in paragraph 1, wherein the catheter body includes an internal passage configured to receive medium through which the longitudinal pressure wave propagates.
3. The catheter system set forth in any one of paragraphs 1-2, wherein the catheter body is configured to deliver the propagated longitudinal pressure wave through the distal end of the catheter body.
4. The catheter system set forth in any one of paragraphs 1-3, wherein the catheter body includes an internal passage filled with a medium through which the longitudinal pressure wave propagates.
5. The catheter system set forth in any one of paragraphs 1-4, further comprising an aspirator configured to remove debris detached from the treatment site during treatment.
6. The catheter system set forth in any one of paragraphs 1-5, further comprising at least one barrier at the distal end portion of the catheter body, wherein the at least one barrier is configured to engage a wall of a body lumen in which the treatment site is dispose to direct the longitudinal pressure wave toward the calcified plaque.
7. The catheter system set forth in paragraph 6, wherein the at least one barrier includes an expandable balloon.
8. The catheter system set forth in paragraph 6, wherein the at least one barrier includes two barriers.
9. The catheter system set forth in paragraph 8, wherein the two barriers are spaced apart from one another such that one of the barriers is a proximal barrier and the other is a distal barrier.
10. The catheter system set forth in paragraph 9, further comprising an extension shaft extending distally outward from the distal end portion of the catheter body, wherein the distal barrier is coupled to the extending shaft.
11. The catheter system set forth in any one of paragraphs 8-10, wherein the two barrier are expandable balloons.
12. The catheter system set forth in paragraph 6, wherein the at least one barrier is configured to limit the longitudinal pressure wave from transmitting therethrough.
13. The catheter system set forth in any one of paragraphs 1-12, further comprising an expandable balloon defining an interior configured to receive fluid, wherein the interior of the balloon is configured to receive the longitudinal pressure wave from the catheter body and direct the longitudinal pressure wave to the calcified plaque at the treatment site.
14. A method of treating calcified plaque at a treatment site within a body of a subject, the method comprising:
   delivering a catheter body of a catheter system to the treatment site so that a distal end portion of the catheter body is adjacent the calcified plaque;
   propagating a longitudinal pressure wave through the catheter body from a proximal end portion toward a distal end portion of the catheter body; and delivering the longitudinal pressure wave from the distal end portion of the catheter body to the calcified plaque.
15. The method set forth in paragraph 14, further comprising inflating a balloon at the distal end portion of the catheter body after said delivering the catheter body and before said propagating a longitudinal pressure wave.
16. The method set forth in any one of paragraphs 14-15, further comprising propagating the longitudinal pressure wave within the inflated balloon before said delivering the longitudinal pressure wave to the calcified plaque.
17. A catheter system for treating calcified plaque within a body of a subject, the catheter system comprising:
   a catheter body having opposite proximal and distal end portion and a longitudinal axis extending therebetween, wherein the catheter body is configured to be percutaneously inserted into the body of the subject and delivered to a treatment site having calcified plaque;
   a treatment member at the distal end portion of the catheter body, the treatment member including a treatment member body and a plurality of fins coupled to the treatment member body, wherein treatment member is selectively configurable between a non-deployed configuration, in which the fins are generally flush with the treatment member body, and a deployed configuration, in which the fins project radially outward from the treatment member body; and
   an actuator configured to enable a user to configure the treatment member between the non-deployed and deployed configurations.
18. The catheter system set forth in paragraph 17, wherein the treatment member body and the fins are integrally formed as a one-piece component, wherein the find are coupled to the treatment member by living hinges.
19. The catheter system set forth in any one of paragraphs 17-18, wherein the actuator is longitudinally movable relative to the treatment member to selectively apply a compressive force on the treatment member to configure the treatment member in the deployed configuration.
20. The catheter system set forth in any one of paragraphs 17-19, wherein the actuator is threadably coupled to the treatment member.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

1-15. (canceled)

16. A catheter system for treating calcified plaque within a body of a subject, the catheter system comprising:
a catheter body having opposite proximal and distal end portion and a longitudinal axis extending therebetween, wherein the catheter body is configured to be percutaneously inserted into the body of the subject and delivered to a treatment site having calcified plaque;
a longitudinal pressure wave generator configured to generate a longitudinal pressure wave, wherein the longitudinal pressure wave generator is couplable to the proximal end portion of the catheter body to enable delivery of the generated longitudinal pressure wave to the catheter body,
wherein the catheter body is configured to enable the longitudinal pressure wave from the longitudinal pressure wave generator to propagate therethough toward the distal end portion of the catheter body and deliver the propagated longitudinal pressure wave to the calcified plaque at the treatment site.

17. The catheter system set forth in claim 16, wherein the catheter body includes an internal passage configured to receive medium through which the longitudinal pressure wave propagates.

18. The catheter system set forth in claim 16, wherein the catheter body is configured to deliver the propagated longitudinal pressure wave through the distal end of the catheter body.

19. The catheter system set forth in claim 16, wherein the catheter body includes an internal passage filled with a medium through which the longitudinal pressure wave propagates.

20. The catheter system set forth in claim 16, further comprising an aspirator configured to remove debris detached from the treatment site during treatment.

21. The catheter system set forth in claim 16, further comprising at least one barrier at the distal end portion of the catheter body, wherein the at least one barrier is configured to engage a wall of a body lumen in which the treatment site is dispose to direct the longitudinal pressure wave toward the calcified plaque.

22. The catheter system set forth in claim 21, wherein the at least one barrier includes an expandable balloon.

23. The catheter system set forth in claim 21, wherein the at least one barrier includes two barriers.

24. The catheter system set forth in claim 23, wherein the two barriers are spaced apart from one another such that one of the barriers is a proximal barrier and the other is a distal barrier.

25. The catheter system set forth in claim 24, further comprising an extension shaft extending distally outward from the distal end portion of the catheter body, wherein the distal barrier is coupled to the extending shaft.

26. The catheter system set forth in claim 24, wherein the two barrier are expandable balloons.

27. The catheter system set forth in claim 21, wherein the at least one barrier is configured to limit the longitudinal pressure wave from transmitting therethrough.

28. The catheter system set forth in claim 16, further comprising an expandable balloon defining an interior configured to receive fluid, wherein the interior of the balloon is configured to receive the longitudinal pressure wave from the catheter body and direct the longitudinal pressure wave to the calcified plaque at the treatment site.

29. A method of treating calcified plaque at a treatment site within a body of a subject, the method comprising:
delivering a catheter body of a catheter system to the treatment site so that a distal end portion of the catheter body is adjacent the calcified plaque;
propagating a longitudinal pressure wave through the catheter body from a proximal end portion toward a distal end portion of the catheter body; and
delivering the longitudinal pressure wave from the distal end portion of the catheter body to the calcified plaque.

30. The method set forth in claim 29, further comprising inflating a balloon at the distal end portion of the catheter body after said delivering the catheter body and before said propagating a longitudinal pressure wave.

31. The method set forth in claim 30, further comprising propagating the longitudinal pressure wave within the inflated balloon before said delivering the longitudinal pressure wave to the calcified plaque.

32. A catheter system for treating calcified plaque within a body of a subject, the catheter system comprising:
a catheter body having opposite proximal and distal end portion and a longitudinal axis extending therebetween, wherein the catheter body is configured to be percutaneously inserted into the body of the subject and delivered to a treatment site having calcified plaque;
a treatment member at the distal end portion of the catheter body, the treatment member including a treatment member body and a plurality of fins coupled to the treatment member body, wherein treatment member is selectively configurable between a non-deployed configuration, in which the fins are generally flush with the treatment member body, and a deployed configuration, in which the fins project radially outward from the treatment member body; and
an actuator configured to enable a user to configure the treatment member between the non-deployed and deployed configurations.

33. The catheter system set forth in claim 32, wherein the treatment member body and the fins are integrally formed as a one-piece component, wherein the find are coupled to the treatment member by living hinges.

34. The catheter system set forth in claim 32, wherein the actuator is longitudinally movable relative to the treatment member to selectively apply a compressive force on the treatment member to configure the treatment member in the deployed configuration.

35. The catheter system set forth in claim 34, wherein the actuator is threadably coupled to the treatment member.
